(19) United States
(12) Patent Application Publication
(10) Pub. No.: US 2024/0317699 A1
(43) Pub. Date: Sep. 26, 2024

(54) A PROCESS FOR THE PURIFICATION OF ANTHOCYANINS AND ANTHOCYANIDINS FROM NATURAL EXTRACTS USING ADSORPTION RESINS AND ACIDIFIED WATER AS DESORBENT

(71) Applicant: AMERICAN BIOPROCESS LIMITADA, Valparaiso (CL)

(72) Inventors: Rodrigo Alejandro MANCILLA VILLALOBOS, Valparaiso (CL); Raul Ignacio ARAVENA CONTRERAS, Santiago (CL)

(21) Appl. No.: 18/574,589

(22) PCT Filed: Jul. 12, 2022
(86) PCT No.: PCT/IB2022/056433

§ 371 Date: Dec. 27, 2023

(30) Foreign Application Priority Data
Jul. 15, 2021 (CL) 1886-2021

(51) Int. Cl. C07D 311/04

(52) U.S. Cl. CPC C07D 311/04

(57) ABSTRACT

A purification process of anthocyanins and anthocyanidins which allows purity increases of at least 3.5 times the initial purity of the extract with yields greater than 50% of recovery, having the following steps: a) contacting the anthocyanin- and anthocyanidin-containing extract with a non-ionic adsorption resin to retain the anthocyanins and anthocyanidins; and b) eluting the resin using water acidified with a monocarboxylic acid at a concentration in the range of 0.01 to 1 M at a temperature between 35 to 100° C., which avoids the use of organic solvents and only uses compounds suitable for human consumption.

### TECHNICAL FIELD

A process for the purification of anthocyanins and anthocyanidins from natural extracts using adsorption resins and acidified water as desorbent is disclosed.

Naturally occurring polyphenols are divided into several sub-groups depending on their molecular structure. Anthocyanidins are an important group of polyphenols found in plants and fruits. Anthocyanidins can be found bound to sugar groups forming a class of compounds known as anthocyanins.

Anthocyanins and anthocyanidins have properties of industrial interest related to health benefits (both for human health and animal feed), strong antioxidant activity, antimicrobial activity, high coloring power, among others. Therefore, currently in the market it is feasible to find multiple products concentrated in these compounds that are marketed in different industries. These products are derived from different sources such as blueberry, cranberry, maqui, elderberry, purple carrot, grape, among others.

Unfortunately, anthocyanins and anthocyanidins are normally found in very low concentrations in plants and fruits, so processes for their extraction, concentration and purification have been developed to take advantage of the benefits of these compounds. For example, the fruit of maqui (Aristotelia chilensis) contains about 0.1% w/w of anthocyanins, while commercial products derived from maqui for the food supplement industry contain over 20% w/w of anthocyanins. A similar situation occurs with blueberry, bilberry and elderberry extracts.

The present patent application proposes a purification process for anthocyanins and anthocyanidins based on adsorption and desorption resins using water acidified with a monocarboxylic organic acid at a pH less than 6. The process allows in one process step to increase the purity of anthocyanins and anthocyanidins at least 3.5 times with respect to the purity of the treated extract. This process has advantages over conventional processes because it avoids the use of organic solvents and only uses compounds suitable for human consumption, which makes the process and product safer by avoiding the use of compounds that are toxic for human consumption. Additionally, avoiding the use of ethanol allows the product obtained from the process to be certified as Halal. From an economic point of view, by using only acidified water without the addition of expensive organic solvents, the process lead to low production costs while high yields (>50%) and purity increases (>3.5 times) are reached.

### STATE OF THE ART

Several methods have been documented using adsorption resins for the purification of anthocyanins and anthocyanidins. For example, U.S. Pat. No. 6,780,442 B2 proposes a method for purifying anthocyanins using brominated resins and elution using ethanol or methanol-containing solutions. A disadvantage of this method is the potential release of bromine during the process, which is a problem for human consumption products. Additionally, this method requires the use of alcohols to elute the anthocyanins from the resin.

Other papers have reported the purification of anthocyanins and/or anthocyanidins from different sources using bromine-free nonionic adsorption resins. However, these papers use some organic solvent, mainly alcohol, in the desorption or elution process. For example, CN107793456 A, this document discloses the purification of anthocyanins from perilla leaves using aqueous ethanol for elution. CN105732741 A, also discloses a process for purifying anthocyanins from perilla leaves using ethanol and methanol in the elution solution. Purification of anthocyanins from purple corn using resins and alcohol as eluent is disclosed in U.S. Pat. No. 7,192,456 B2. CN105859674 A discloses anthocyanins purification from dragon fruit peel using aqueous methanol for elution. U.S. Pat. No. 6,461,648 B2 proposes a method for purifying anthocyanidins and anthocyanins without limitation to a raw material using adsorption resin but requiring the use of an alcoholic solution as eluent. CN102875515 B discloses a method for purifying anthocyanins from blueberry using aqueous solutions of methanol and ethanol as desorbent and adding formic acid or acetic acid to the desorbent mixture.

A different process using ion exchange resins is proposed in EP 1443948 B1, but the process still requires the use of some alcohol for elution. Similarly, U.S. Pat. No. 8,575,334 B2 proposes a method for purifying anthocyanins using cation exchange resins and an alkaline solution that would promote desorption of the anthocyanins. A problem with this method is that alkaline conditions promote rapid anthocyanin degradation affecting anthocyanin recovery. Additionally, the method suggests the use of alcohols in the elution solvent.

Document CN110372659 A, discloses blueberry anthocyanins recovery and their purification using a resin adsorption step and subsequent elution with citric acid. However, purities up to 8% can be reached using the process, while the process disclosed by the present patent application leads to much higher purities as can be observed in the examples.

In summary, none of the documents cited as prior art discloses a simple process which leads in one process step to increase at least 3.5 times the purity of anthocyanins and anthocyanidins in comparison to the purity of the treated extract. The process of the present invention also has advantages over conventional processes in that it avoids the use of organic solvents and uses only compounds suitable for human consumption, which makes the process and product safer by avoiding the use of compounds toxic to human consumption.

### DESCRIPTION OF THE DRAWINGS

FIG. 1: This figure corresponds to a chromatogram showing the anthocyanin profile of a purified maqui extract obtained by this method.

FIG. 2: This figure corresponds to a chromatogram showing the anthocyanin profile of a purified calafate extract obtained by this method.

FIG. 3: This figure corresponds to a chromatogram showing the anthocyanin profile of a purified elderberry extract obtained by this method.

### DESCRIPTION OF THE INVENTION

The present invention discloses a process of purification of anthocyanins and anthocyanidins based on the use of adsorption resins using acidified water for desorption. The process described in the present patent application makes it possible to obtain in a single purification step purity increments of at least 3.5 times the initial purity of the extract with yields greater than 50% recovery.

The process of the present invention comprises the following steps:
(a) contacting the extract containing anthocyanins and anthocyanidins with a nonionic adsorption resin to retain the anthocyanins and anthocyanidins, and
b) eluting the resin using hot water acidified with a monocarboxylic acid.

In step a) the nonionic adsorption resin can be any commercially available one. Non-ionic resins that can be used, for example, but not limited to these, are XAD4, XAD7, XAD16, XAD18, XAD1180, XAD1600, FPX66, AB-8, D101, LS305, LX-60, LX-68, PAD400, PAD900, PAD610, PAD950, X5, H103, among others.

The term monocarboxylic acid refers to any carboxylic acid with a single carboxyl group and <10 carbons with the formula (COOH)CₓH_{y}, where ‘x’ at most is 9 and “y” is equal to or greater than 0. Acids that belong to this category are formic acid, acetic acid, propionic acid, butyric acid, pentanoic acid, hexanoic acid, heptanoic acid, octanoic acid, among others. Dicarboxylic acids or carboxylic acids with other functional groups (i.e., alcohol) do not lead to high purity end products as shown in Example 1. Surprisingly, propionic acid shows the best results in terms of purity and recovery with respect to the other monocarboxylic acids.

In step b) the concentration of monocarboxylic acid is preferably in the range of 0.01 to 1 M, and more preferably in the range of 0.05 to 0.9 M to obtain higher purities. The low concentrations of monocarboxylic acid required in this process present advantages in terms of economics over other processes that require high levels of organic solvents to be performed.

Optionally, a mixture of monocarboxylic acids can be used in step (b).

The elution temperature in stage (b) can be anywhere between 35 and 100° C., and more preferably in the range of 40° C. to 90° C.

The anthocyanin-purified extract obtained in (b) has at least 3.5 times the purity of the original extract contacted in (a), and more preferably between 4 to 10 times the purity of the extract contacted in (a). By the process of the present patent application after elution at least 50% of the anthocyanins present in the initial extract are recovered.

A batch or continuous system can be used to carry out the process. Continuous systems are preferred, as they require less process times for the same amount of resin used. In addition, continuous systems allow that part of the volume collected at the outlet of the system, which has low anthocyanin and anthocyanidin purity, can be easily discarded to further increase the purity of the extract, while batch systems would require multiple desorption steps to obtain a similar result.

Optionally, the resin obtained after the elution of anthocyanins and anthocyanidins in step (b) is regenerated using an aqueous alkaline solution at a temperature below 100° C. More preferably, the resin can be regenerated with an aqueous solution with 1-5% w/w of NaOH, KOH, Ca(OH)₂, Na₂CO₃, NH₄OH or mixtures thereof at a temperature below 100° C. The regenerated resin can be reused for a new purification cycle.

Optionally, the eluted extract obtained from step (b) purified into anthocyanins and anthocyanidins can be concentrated to remove the solvent. Any method known from the state of the art can be used to perform the concentration. Methods such as falling film evaporation, rising film evaporation, scraped film evaporation, nanofiltration, reverse osmosis, pervaporation, among others, can be used.

Optionally, the eluted extract obtained from step (b) or its concentrated product can be dried to obtain a powder. The drying method can be any known in the state of the art such as spray drying and freeze drying.

### EXAMPLES

### Example 1

Maqui extracts were contacted with FPX66 resin in a stainless-steel reactor for 2 hours at 25° C. with constant agitation at 150 RPM.

The resin was placed in a basket attached to the stirrer shaft to facilitate its separation from the extract. After the adsorption step, the extract was removed from the reactor and the desorbent solution was loaded. Desorption was carried out at 450 RPM for 20 minutes using different acids to work with acidified water. Table I, indicates details of the different tests performed.

TABLE I

| | Extract | | Adsorption | | Desorption | | | | |
|---|---|---|---|---|---|---|---|---|---|
| | ACN¹ | Purity² | ACN³ | | | ACN⁵ | Purity³ | | Conc. |
| | (mg/L) | ACN (%) | ads (%) | R/E⁴ | T(° C.) | rec (%) | ACN (%) | Acid | Ac⁶ (M) |
| 1 | 894 | 1.9 | 77.5 | 61 | 80 | 52.2 | 14.8 | propionic | 0.2 |
| 2 | 769 | 4.8 | 74.9 | 43 | 80 | 61.1 | 29.9 | acetic | 0.05 |
| 3 | 869 | 5.8 | 74.0 | 52 | 80 | 66.2 | 26.7 | acetic | 0.2 |
| 4 | 855 | 5.5 | 66.4 | 50 | 80 | 67.2 | 20.3 | acetic | 1.00 |
| 5 | 906 | 5.5 | 78.4 | 54 | 80 | 67.7 | 29.8 | propionic | 0.05 |
| 6 | 842 | 5.5 | 71.0 | 50 | 80 | 71.8 | 27.5 | propionic | 0.20 |
| 7 | 855 | 5.5 | 67.8 | 51 | 80 | 73.3 | 20.5 | propionic | 1.00 |
| 8 | 3,306 | 6.7 | 91.3 | 220 | 80 | 74.1 | 22.8 | propionic | 0.40 |
| 9 | 797 | 4.9 | 69.7 | 47 | 80 | 71.4 | 15.5 | butyric | 0.20 |
| 10 | 848 | 5.0 | 77.7 | 57 | 80 | 65.4 | 19.2 | octanoic | 0.20 |
| 11 | 769 | 4.8 | 74.9 | 43 | 30 | 43.2 | 29.5 | propionic | 0.20 |
| 12 | 769 | 4.8 | 74.9 | 43 | 40 | 56.7 | 29.1 | propionic | 0.20 |
| 13 | 769 | 4.8 | 74.9 | 43 | 50 | 61.1 | 29.9 | propionic | 0.20 |
| 14 | 769 | 4.8 | 74.9 | 43 | 60 | 66.0 | 28.7 | propionic | 0.20 |
| 15 | 769 | 4.8 | 74.9 | 43 | 70 | 68.8 | 28.0 | propionic | 0.20 |
| 16 | 769 | 4.8 | 74.9 | 43 | 90 | 64.4 | 23.6 | propionic | 0.20 |
| 17 | 855 | 5.2 | 70.4 | 50 | 110 | 42.0 | 16.3 | propionic | 0.20 |
| 18 | 848 | 5.0 | 79.3 | 53 | 80 | 65.5 | 2.6 | lactic | 0.16 |
| 19 | 791 | 5.1 | 66.8 | 47 | 80 | 68.8 | 0.8 | oxalic | 0.20 |
| 20 | 791 | 5.1 | 71.0 | 47 | 80 | 63.2 | 0.5 | succinic | 0.20 |
| 21 | 935 | 5.6 | 78.8 | 52 | 80 | 51.1 | 0.3 | citric | 0.20 |
| 22 | 935 | 5.6 | 77.9 | 52 | 80 | 67.4 | 18.0 | acetic | 4.37 |

¹Initial concentration of anthocyanins and anthocyanidins (ACN).
²Purity of anthocyanins and anthocyanidins on dry basis (d.b).
³Quantity of anthocyanins and anthocyanidins adsorbed with respect to those contained in the extract.
⁴Ratio of resin to extract used for adsorption.
⁵Recovery of anthocyanins after desorption with respect to the adsorbed anthocyanins and anthocyanidins.
⁶Concentration of acid used in the desorption solution.

Test 1 was carried out using blueberry extract, while the other tests corresponded to maqui extract. On the other hand, test 8 was carried out with a maqui extract previously concentrated in 5 kDa ultrafiltration membranes. In the case of the test with octanoic acid, and due to the low solubility of this acid in water, the mixture recovered from the reactor was allowed to cool to room temperature and the aqueous phase was recovered. In other cases only one aqueous phase was obtained. It is worth mentioning that similar results were observed using XAD4 and XAD7HP resins.

From the results obtained in these tests, it can be observed that the use of acidified water using monocarboxylic acids (1 to 17) results in final extracts with higher purities than other carboxylic acids (18 to 21). It is worth mentioning that tests using phosphoric acid and hydrochloric acid were also performed, obtaining purities lower than those of the fed extract. Among the monocarboxylic acids, propionic acid is the one that shows the best results with purities as high as acetic acid, but with higher recovery yields. Therefore, the use of propionic acid has an advantage over other acids.

On the other hand, test 6 and tests 11 to 17 show the effect of temperature on the desorption process using acidified water. From the results, an increase in temperature negatively affects purity in the studied range, being the effect stronger at temperatures above 90° C. On the other hand, recovery is positively affected by temperature up to 80° C., after which the recovery of anthocyanins and anthocyanidins begins to decrease.

When acetic acid and propionic acid at different concentrations were used, it was observed that the purity decreases when the acid concentration increases. In fact, in Example 22, it is observed that it is not possible to achieve a 3.5-fold increase in purity over the initial purity of the extract when an excess of acetic acid was used.

Finally, example 22 shows that an acid concentration above that suggested in this invention does not improve the recovery of anthocyanins and anthocyanidins, it has a detrimental effect on purity (less than 3.5-fold increase) and increases the cost of the process by requiring very high amounts of acid.

### Example 2

A maqui extract with an initial anthocyanin and anthocyanidin concentration of 835.8 mg/L, and an anthocyanin and anthocyanidin purity of 5.6% (d.b) was processed in a continuous bed loaded with 45.0 g of FPX66 resin. To load the resin, 684.4 mL of extract were treated with the resin at room temperature. After the adsorption process was completed, the resin was desorbed using 660 mL of water acidified with 0.85 M propionic acid at 80° C. Table II shows the anthocyanins recovery and the purity of the accumulated extract:

TABLE II

| Fractions | Time (min) | Purity¹ (%) | Recovery² (%) |
|---|---|---|---|
| | 0 | 0 | — |
| 1 | 7.9 | 0.1 | 0.1 |
| 2 | 18.0 | 10.1 | 18.2 |
| 3 | 28.1 | 17.8 | 49.7 |
| 4 | 38.0 | 20.2 | 65.2 |
| 5 | 49.4 | 20.4 | 69.5 |
| 6 | 70.0 | 20.6 | 73.5 |
| 7 | 90.1 | 20.5 | 74.8 |
| 8 | 112.6 | 20.4 | 75.6 |
| 9 | 140.2 | 20.3 | 76.2 |
| 10 | 150 | 20.2 | 76.3 |

¹Purity of anthocyanins and anthocyanidins on a dry basis (d.b).
²Recovery of anthocyanins and anthocyanidins with respect to extract content.

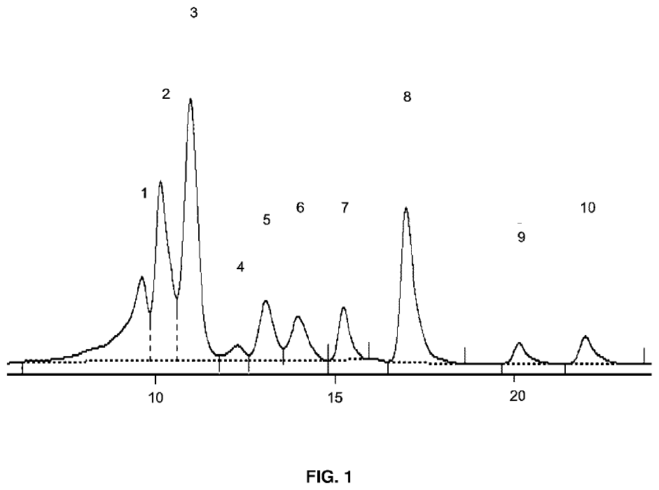

The cumulative of all fractions collected reached a total purity of 20.2% (d.b) anthocyanins and anthocyanidins with a recovery of 76.3% of the anthocyanins and anthocyanidins contained in the original extract, which is an improvement of −3.6 times in the anthocyanin purity. The chromatogram in FIG. 1 shows the anthocyanin profile of the extract obtained by this method.

TABLE III

| Peak | Compound |
|---|---|
| 1 | unknown |
| 2 | Delphinidin-3-O-samboside-5-O-glucoside |
| 3 | Delphinidin 3,5-O diglucoside |
| 4 | unknown |
| 5 | Cyanidine-3-O-samboside-5-O-glucoside |
| 6 | Cyanidine 3,5-O diglucoside |
| 7 | Delphinidin-3-O-samboside |
| 8 | Delphinidin-3-O-glucoside |
| 9 | Cyanidine-3-O-samboside |
| 10 | Cyanidine-3-O-glucoside |

To compare the use of acetic acid with propionic acid, a maqui extract with an initial anthocyanin and anthocyanidin concentration of 841.9 mg/L and an anthocyanin and anthocyanidin purity of 5.2% (d.b) was processed in a continuous bed loaded with 40.5 g of FPX66 resin. To load the resin, 596 mL of extract were treated with the resin at room temperature. After the adsorption process was completed, the resin was desorbed using 540 mL of water acidified with 0.85 M acetic acid at 80° C. Table Ill shows the anthocyanins recovery and the purity of the accumulated extract:

TABLE IV

| Fractions | Time (min) | Purity¹ (%) | Recovery² (%) |
|---|---|---|---|
| | 0 | 0 | — |
| 1 | 11.0 | 0.03 | 0.0 |
| 2 | 21.2 | 3.07 | 5.4 |
| 3 | 31.5 | 8.20 | 18.1 |
| 4 | 41.6 | 12.12 | 29.5 |
| 5 | 54.1 | 14.84 | 38.7 |
| 6 | 72.1 | 17.23 | 47.7 |
| 7 | 90.0 | 18.49 | 53.2 |
| 8 | 110.0 | 19.21 | 56.9 |
| 9 | 122.7 | 19.48 | 58.4 |
| 10 | 135.0 | 19.70 | 59.7 |

¹Purity of anthocyanins and anthocyanidins on a dry basis (d.b).
²Recovery of anthocyanins and anthocyanidins with respect to extract content.

The cumulative of all fractions collected reached a total purity of 19.7% (d.b) anthocyanins and anthocyanidins with a recovery of 59.7% of the anthocyanins and anthocyanidins contained in the original extract, which shows an improvement of −3.7 times in the anthocyanins and anthocyanidins purity. As observed in Example 1, the use of acetic acid generates a final extract with a similar purity to propionic acid, but lower recovery yields. Therefore, propionic acid is preferred if one seeks to optimize the process in terms of yield.

Finally, the purification of the same extract used for the acetic acid test was evaluated but using hot non-acidified water at 80° C. (distilled water). An extract with a purity of 12.4% (d.b) was obtained and a recovery of 26.6% of the anthocyanins and anthocyanidins was reached, which shows the benefit of using acidified water with a mono-carboxylic acid in the desorption.

When comparing a desorption using hot acidulated water (80° C.) with a desorption using ethanol-water (50% w/w) at room temperature, higher purity and recovery of anthocyanins and anthocyanidins were observed with hot acidulated water. When the temperature of the ethanol-water desorption mixture (50%) was increased to 60° C., the recovery of anthocyanins and anthocyanidins increased to a value close to that of using acidified hot water, but a reduction in the purity of anthocyanins and anthocyanidins was observed. This result is surprising since aqueous solutions of alcohols, and particularly ethanol, are widely described in the literature as desorption solvents to purify anthocyanins using resin adsorption processes.

### Example 3

A calafate extract with an initial anthocyanin and anthocyanidin concentration of 1203.6 mg/L and an anthocyanin and anthocyanidin purity of 8.7% (d.b) was processed in a continuous bed loaded with 43.0 g of FPX66 resin. To load the resin, 446.3 mL of extract were treated, reaching an adsorption of 99.7% of the anthocyanins and anthocyanidins contained in the extract. Once the adsorption process was completed, the resin was desorbed using 582 mL of water acidified with 0.85 M propionic acid at 80° C. Table IV shows the anthocyanins recovery and the purity of the accumulated extract:

TABLE V

| Fractions | Time (min) | Purity¹ (%) | Recovery² (%) |
|---|---|---|---|
| | 0 | — | 0 |
| 1 | 10.6 | 0 | 0 |
| 2 | 23.2 | 5.1 | 6.06 |
| 3 | 34.5 | 14.8 | 24.50 |
| 4 | 45.8 | 22.2 | 42.04 |
| 5 | 58.5 | 25.4 | 51.37 |
| 6 | 70.4 | 27.0 | 56.60 |
| 7 | 92.1 | 28.8 | 60.65 |
| 8 | 112.1 | 29.1 | 62.64 |
| 9 | 124.6 | 30.2 | 63.47 |
| 10 | 138.5 | 31.3 | 64.29 |

¹Purity of anthocyanins and anthocyanidins on dry basis (d.b).
²Recovery of anthocyanins and anthocyanidins with respect to anthocyanins and anthocyanidins adsorbed on resin.

The cumulative of all fractions collected reached a total purity of 28.3% (d.b) anthocyanins and anthocyanidins and a recovery of 64.29% of the adsorbed anthocyanins and anthocyanidins (63.6% total anthocyanin recovery), showing a ˜3.6-fold improvement in the anthocyanin purity in comparison to the initial extract.

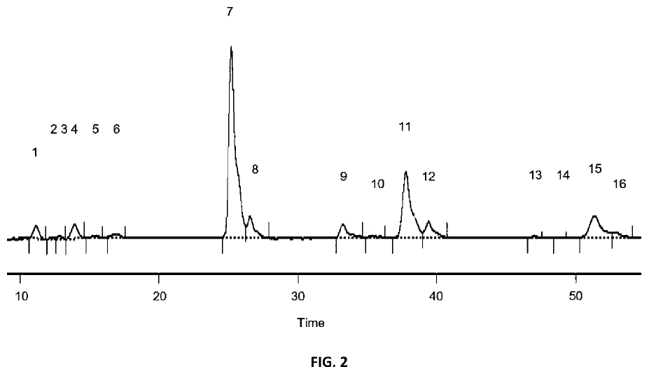

The chromatogram in FIG. 2 shows the anthocyanin profile of the extract obtained by this method.

TABLE VI

| Peak | Compound |
|---|---|
| 1 | Delphinidine-3,5-dihexoside |
| 2 | Delphinidin-3-rutinoside-5-glucoside |
| 3 | Cyanidin-3,5-dihexoside |
| 4 | Petunidin-3,5-dihexoside |
| 5 | Petunidin-3-rutinoside-5-glucoside |
| 6 | Peonidin-3,5-dihexoside |
| 7 | Delphinidin-3-glucoside |
| 8 | Delphinidine-3-rutinoside |
| 9 | Malvidin-3-rutinoside-5-glucoside |
| 10 | Cyanidin-3-glucoside |
| 11 | Cyanidin-3-rutinoside |
| 12 | Petunidin-3-glucoside |
| 13 | Petunidin-3-rutinoside |
| 14 | Peonidin-3-glucoside |
| 15 | Peonidin-3-rutinoside |
| 16 | Malvidin-3-glucoside |

### Example 4

An elderberry extract with an initial anthocyanin and anthocyanidin concentration of 1004.4 mg/L and an anthocyanin and anthocyanidin purity of 1.9% (d.b) was processed in a continuous bed loaded with 45.2 g of FPX66 resin. To load the resin, 796.4 mL of extract were treated and 99.3% of the anthocyanins and anthocyanidins contained in the extract were adsorbed. Once the adsorption process was completed, the resin was desorbed using 792 mL of water acidified with 0.85 M propanoic acid at 80° C. Table V shows the anthocyanins recovery and the purity of the accumulated extract:

TABLE VII

| Fractions | Time (min) | Purity¹ (%) | Recovery² (%) |
|---|---|---|---|
| | 0 | — | 0 |
| 1 | 9.0 | 0.28 | 0.59 |
| 2 | 20.0 | 1.74 | 7.34 |
| 3 | 31.0 | 3.82 | 20.59 |
| 4 | 41.4 | 5.12 | 30.61 |
| 5 | 51.2 | 5.74 | 36.36 |
| 6 | 90.0 | 7.06 | 50.06 |
| 7 | 108.4 | 7.32 | 53.23 |
| 8 | 150.8 | 7.59 | 57.02 |
| 9 | 171.4 | 7.64 | 58.02 |
| 10 | 180.0 | 7.66 | 58.38 |

¹Purity of anthocyanins and anthocyanidins on dry basis (d.b).
²Recovery of anthocyanins and anthocyanidins with respect to anthocyanins and anthocyanidins adsorbed on resin.

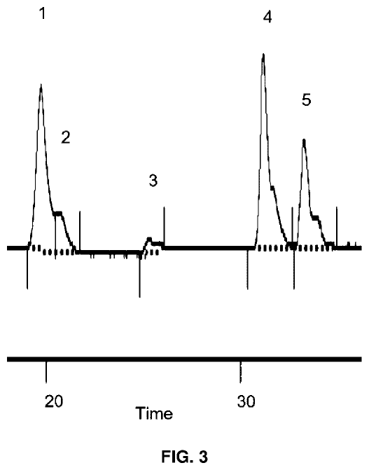

The cumulative of all fractions collected reached a total purity of 7.6% (d.b) anthocyanins and anthocyanidins and a recovery of 58.3% of the adsorbed anthocyanins and anthocyanidins (57.9% total anthocyanin recovery), showing a ˜4-fold improvement in anthocyanin purity in comparison to the initial extract. The chromatogram in FIG. 3 shows the anthocyanin profile of the extract obtained by this method.

TABLE VIII

| Peak | Compound |
|---|---|
| 1 | Cyanidin-3,5-O-diglucoside |
| 2 | Delphinidin-3-O-sambioside |
| 3 | Delphinidin-3-glucoside |
| 4 | Cyanidin-3-O-sambioside |
| 5 | Cyanidin-3-O-glucoside |

### Example 5

A purified maqui extract obtained as described in Example 2 (purity of 21.1% (d.b) of anthocyanins and anthocyanidins) was concentrated in a vacuum wiped film evaporator at 60° C. After the evaporation, a concentrated extract contained 25.4% solids with a purity of 19.6% (d.b) of anthocyanins and anthocyanidins. Then, the concentrated extract was mixed with 10% maltodextrin and 1% silicon dioxide to be dried in a spray dryer at an inlet temperature of 120° C. and an outlet temperature of 70° C. The final product was a powder with a purity of anthocyanins and anthocyanidins of 15.6% (d.b) and a humidity of 2.2%.

1. Anthocyanins and anthocyanidins purification process which reaches over 50% of recovery, the process comprises the following steps:
a) contacting the extract containing anthocyanins and anthocyanidins with a nonionic adsorption resin to retain the anthocyanins and anthocyanidins; and
b) eluting the resin using water acidified with a monocarboxylic acid at a concentration in the range of 0.01 to 1 M at a temperature between 35 to 100° C.
wherein the monocarboxylic acid is any carboxylic acid with only one carboxylic group and containing <10 carbons based on the formula (COOH)CxHy, in which x is maximum 9 and y is higher than 0.

2. The process according to claim 1, wherein the nonionic adsorption resin is selected from the group consisting of XAD4, XAD7, XAD16, XAD18, XAD1180, XAD1600, FPX66, AB-8, D101, LS305, LX-60, LX-68, PAD400, PAD900, PAD610, PAD950, X5 and H103.

3. The process according to claim 1, wherein the monocarboxylic acid used in step b) is selected from formic acid, acetic acid, propionic acid, butyric acid, pentanoic acid, hexanoic acid, heptanoic acid, octanoic acid or a mixture thereof.

4. The process according to claim 3, wherein the monocarboxylic acid used in step b) is propionic acid.

5. The process according to claim 1, wherein in step b) the concentration of monocarboxylic acid is in the range of 0.05 to 0.9 M.

6. The process according to claim 1, wherein the temperature in stage (b) is in the range of 40° C. to 90° C.

7. The process according to claim 1, wherein a batch or continuous mode system is used.

8. The process according to claim 7, wherein a continuous mode system is used.

9. The process according to claim 1, wherein the resin obtained after the elution of anthocyanins and anthocyanidins in step (b) is regenerated using an alkaline aqueous solution at a temperature lower than 100° C.

10. The process according to claim 9, wherein the resin can be regenerated with an aqueous solution containing 1-5% NaOH, KOH, Ca(OH)₂, Na₂CO₃, NH₄OH or mixtures thereof at a temperature below 100° C.

11. The process according to claim 1, wherein the eluted extract obtained from step (b) purified in anthocyanins and anthocyanidins can be concentrated for solvent removal.

12. The process according to claim 11, wherein the concentration of the eluted extract obtained in step (b) is obtained by falling film evaporation, rising film evaporation, scraped film evaporation, nanofiltration, reverse osmosis or pervaporation.

13. The process according to claim 1, wherein the eluted extract obtained from step (b), or its concentrated product can be dried to obtain a powdered product.

14. The process according to claim 13, wherein the eluted extract obtained from step (b) or its concentrated product is spray dried and freeze dried.

15. A product obtained based on the process according to claim 13.

16. (canceled)

17. (canceled)

18. (canceled)
